SEQUENCE LISTING (8 sequences)

SEQ ID NO: 1 - protein, 424 aa, synthetic construct
MTLFATASTVAICSYALLSTVYKTAQVFYTLPTNVPPTSGDPPGGEPWPSVDVIIPCYNEAPRTLSDCLASIASQEYAGKLQVYVVDDGSANRDALVGVHEEYAGDPRFNFIALPKNVGKRKAQIAAIRRSCGDLVLNVDSDTILAPDVITRLALKMQDQAVGAAMGQLAASNRSETWLTRLIDMEYWLACNEERAAQARFGAVMCCCGPCAMYRRSALVSLLDQYETQRFRGKPSDFGEDRHLTILMLKAGFRTEYVPEAVAATVVPNSMGPYLRQQLRWARSTFRDTLLAFQLLRGLNIYLTLDVIGQNIGPLLLSLSILAGLAQLVTTGTAPWTACLMIAAMTIVRCSVAAFRARQLRFLGFSLHTLINIFLLLPLKAYALCTLSNSDWLSRSSAANVTDTGGTSSKPNLVGSDAAYSEQQ

SEQ ID NO: 2 - DNA, 1275 bp, synthetic construct
  source 1..1275: mol_type = other DNA
atgaccctgtttgcaaccgcaagcaccgttgcaatttgtagctatgcactgctgagcaccgtttataaaaccgcacaggttttttataccctgccgaccaatgttccgccgaccagcggtgatcctccgggtggtgaaccttggcctagtgttgatgttattattccgtgttataacgaagccccgcgtaccctgagcgattgtctggcaagtattgcaagccaggaatatgcaggtaaactgcaggtttatgttgtggacgatggtagcgcaaatcgtgatgcactggttggtgttcatgaagaatatgcaggtgatccgcgttttaattttatcgcactgccgaaaaatgtgggcaaacgtaaagcacagattgccgcaattcgtcgtagctgtggtgatctggttctgaatgttgatagcgataccattctggcaccggatgttattacccgtctggcactgaaaatgcaggatcaggcagttggtgcagcaatgggtcagctggcagcaagcaatcgtagcgaaacctggctgacccgtctgattgatatggaatattggctggcatgtaatgaggaacgtgcagcacaggcacgttttggtgcagttatgtgttgttgtggtccgtgtgcaatgtatcgtcgtagcgcactggttagcctgctggatcagtatgaaacccagcgttttcgtggtaaaccgagcgattttggtgaagatcgtcatctgaccattctgatgctgaaagcaggttttcgtaccgaatatgttccggaagcagttgcagcaaccgttgttccgaatagcatgggtccgtatctgcgtcagcagctgcgttgggcacgtagcacatttcgtgataccctgctggcatttcagctgctgcgtggtctgaatatttatctgaccctggatgttattggccagaatattggtccgctgctgctgagcctgagcattctggcaggtctggcacagctggttaccaccggtaccgcaccttggacagcatgtttaatgattgcagcaatgaccattgtgcgctgtagcgttgcagcatttcgtgcacgtcagctgcgttttctgggttttagcctgcataccctgattaatattttcctgctgctgccgctgaaagcatatgcactgtgtaccctgagcaatagcgattggctgagccgtagcagcgcagcaaatgttaccgataccggtggtaccagcagcaaaccgaatctggttggtagcgatgcagcatatagcgaacagcagtga

SEQ ID NO: 3 - DNA, 35 bp, synthetic construct
  source 1..35: mol_type = other DNA
cgtgtcaaagaaattattgaatcacaaaaaacagg

SEQ ID NO: 4 - DNA, 23 bp, synthetic construct
  source 1..23: mol_type = other DNA
gcatgtgggtgatgtagatctcg

SEQ ID NO: 5 - protein, 424 aa, synthetic construct
MTLFATASTVAICSYALLSTVYKTAQVFYTLPTNVPPTSGDPPGGEPWPSVDVIIPCYNEAPRTLSDCLASIASQEYAGKLQVYVVDDGSANRDALVGVHEEYAGDPRFNFIALPKNVGKRKAQIAAIRRSCGDLVLNVDSDTILAPDVITRLALKMQDQAVGAAMGQLAASNRSETWLTRLIDMEYWLACNEERAAQARFGAVMCCCGPCAMYRRSALVSLLDQYETQRFRGKPSDFGEDRHLTILMLKAGFRTEYVPEAVAATVVPNSMGPYLRQQLRWARSTFRDTLLAFQLLRGLNIYLTLDVIGQNIGPLLLSLSILAGLAQLVTTGTAPWTACLMIAAMTIVHCSVAAFRARQLRFLGFSLHTLINIFLLLPLKAYALCTLSNSDWLSRSSAANVTDTGGTSSKPNLVGSDAAYSEQQ

SEQ ID NO: 6 - DNA, 1275 bp, synthetic construct
  source 1..1275: mol_type = other DNA
atgaccctgtttgcaaccgcaagcaccgttgcaatttgtagctatgcactgctgagcaccgtttataaaaccgcacaggttttttataccctgccgaccaatgttccgccgaccagcggtgatcctccgggtggtgaaccttggcctagtgttgatgttattattccgtgttataacgaagccccgcgtaccctgagcgattgtctggcaagtattgcaagccaggaatatgcaggtaaactgcaggtttatgttgtggacgatggtagcgcaaatcgtgatgcactggttggtgttcatgaagaatatgcaggtgatccgcgttttaattttatcgcactgccgaaaaatgtgggcaaacgtaaagcacagattgccgcaattcgtcgtagctgtggtgatctggttctgaatgttgatagcgataccattctggcaccggatgttattacccgtctggcactgaaaatgcaggatcaggcagttggtgcagcaatgggtcagctggcagcaagcaatcgtagcgaaacctggctgacccgtctgattgatatggaatattggctggcatgtaatgaggaacgtgcagcacaggcacgttttggtgcagttatgtgttgttgtggtccgtgtgcaatgtatcgtcgtagcgcactggttagcctgctggatcagtatgaaacccagcgttttcgtggtaaaccgagcgattttggtgaagatcgtcatctgaccattctgatgctgaaagcaggttttcgtaccgaatatgttccggaagcagttgcagcaaccgttgttccgaatagcatgggtccgtatctgcgtcagcagctgcgttgggcacgtagcacatttcgtgataccctgctggcatttcagctgctgcgtggtctgaatatttatctgaccctggatgttattggccagaatattggtccgctgctgctgagcctgagcattctggcaggtctggcacagctggttaccaccggtaccgcaccttggacagcatgtttaatgattgcagcaatgaccattgtgcattgtagcgttgcagcatttcgtgcacgtcagctgcgttttctgggttttagcctgcataccctgattaatattttcctgctgctgccgctgaaagcatatgcactgtgtaccctgagcaatagcgattggctgagccgtagcagcgcagcaaatgttaccgataccggtggtaccagcagcaaaccgaatctggttggtagcgatgcagcatatagcgaacagcagtga

SEQ ID NO: 7 - DNA, 30 bp, synthetic construct
  source 1..30: mol_type = other DNA
cattgtgcattgtagcgttgcagcatttcg

SEQ ID NO: 8 - DNA, 31 bp, synthetic construct
  source 1..31: mol_type = other DNA
caacgctacaatgcacaatggtcattgctgc

(19) United States
(12) Patent Application Publication
(10) Pub. No.: US 2026/0275303 A1
(43) Pub. Date: Sep. 17, 2026

(54) N-ACETYLGLUCOSAMINYLTRANSFERASE AcetRL-R349H AND APPLICATION THEREOF

(71) Applicant: OCEAN UNIVERSITY OF CHINA, Qingdao City (CN)

(72) Inventors: Jianan SUN, Qingdao City (CN); Qiang ZHAO, Qingdao City (CN); Xiangzhao MAO, Qingdao City (CN); Haipeng SU, Qingdao City (CN); Yongzhen WANG, Qingdao City (CN)

(21) Appl. No.: 19/400,122

(22) Filed: Nov. 25, 2025

(30) Foreign Application Priority Data
Mar. 14, 2025 (CN) 202510299689.0

(51) Int. Cl. C12N 9/10; C12P 19/04; C12P 19/18

(52) U.S. Cl. CPC C12N 9/1051; C12P 19/04; C12P 19/18

(57) ABSTRACT

The present invention discloses an N-acetylglucosaminyltransferase AcetRL-R349H and an application thereof, belonging to the technical field of functional enzymes. An amino acid sequence of the N-acetylglucosaminyltransferase AcetRL-R349H is as shown in SEQ ID NO. 5. An application of the N-acetylglucosaminyltransferase AcetRL-R349H in preparation of CTOS. The N-acetylglucosaminyltransferase AcetRL-R349H of the present invention is obtained by performing mutation modification on an N-acetylglucosaminyltransferase AcetRL, and can be used to produce the CTOS with a degree of polymerization of 4-6, especially (GlcNAc)₆. The N-acetylglucosaminyltransferase AcetRL-R349H and the application thereof provided by the present invention broaden a product spectrum of N-acetylglucosaminyltransferases, can be used to prepare the CTOS with a high degree of polymerization, and have broad application prospects.

### REFERENCE TO AN ELECTRONIC SEQUENCE LISTING

The content of the electronic sequence listing (2025-11-25-SequenceListing.xml; Size: 11,252 bytes; and Date of Creation: 2025 Aug. 19) is herein incorporated by reference in its entirety.

### TECHNICAL FIELD

The present invention relates to an N-acetylglucosaminyltransferase AcetRL-R349H and an application thereof, belonging to the technical field of functional enzymes.

### BACKGROUND

Chitin oligosaccharides (CTOS) are functional oligosaccharides with a degree of polymerization of 2-10, formed by linkage of N-acetylglucosamine (GlcNAc) via β-1,4 glycosidic bonds. CTOS have biological activities including antioxidation, anti-tumor, prevention of atherosclerosis, and promotion of plant growth. The degree of polymerization of CTOS has a great impact on their biological activities.

N-acetylglucosaminyltransferase is a glycosyltransferase that can produce CTOS using GlcNAc and uridine diphosphate-N-acetylglucosamine (UDP-GlcNAc) as substrates. However, N-acetylglucosaminyltransferases in the prior art can only produce CTOS with a degree of polymerization of 3-5. The degree of polymerization of products is highly limited, making it difficult to achieve the production of CTOS with a degree of polymerization greater than 5. Therefore, based on the N-acetylglucosaminyltransferase, obtaining N-acetylglucosaminyltransferases that have an ability to produce CTOS with a higher degree of polymerization through rational design strategies has broad application prospects.

### SUMMARY

In view of the above-mentioned prior art, the present invention provides an N-acetylglucosaminyltransferase AcetRL-R349H and an application thereof, belonging to the technical field of functional enzymes.

The present invention is achieved by the following technical solution:

An N-acetylglucosaminyltransferase AcetRL-R349H, where an amino acid sequence of the N-acetylglucosaminyltransferase AcetRL-R349H is as shown in SEQ ID NO. 5.

An application of the N-acetylglucosaminyltransferase AcetRL-R349H in preparation of CTOS.

Further, the CTOS are any one or two or more of chitotetraose, chitopentaose, or chitohexaose.

Further, in specific applications, a coding gene of the N-acetylglucosaminyltransferase AcetRL-R349H is introduced into Bacillus subtilis to construct a recombinant engineering bacterium expressing the N-acetylglucosaminyltransferase AcetRL-R349H; and the recombinant engineering bacterium is fermented to obtain a fermentation broth containing the CTOS. A principle for producing the CTOS is as follows: by using GlcNAc and UDP-GlcNAc produced by Bacillus subtilis itself as substrates, the CTOS are prepared under action of the N-acetylglucosaminyltransferase AcetRL-R349H.

Further, the Bacillus subtilis is a Bacillus subtilis strain 168. The Bacillus subtilis strain 168 is a Gram-positive, rod-shaped bacterium, which is commonly used as a model organism in molecular biology and genetics. It is the most extensively studied strain among Bacillus subtilis, and its genome has been extensively sequenced and annotated.

The N-acetylglucosaminyltransferase AcetRL-R349H of the present invention is obtained by performing mutation modification on an N-acetylglucosaminyltransferase AcetRL, has product specificity after the mutation modification, and can be used to produce the CTOS with a degree of polymerization of 4-6, especially (GlcNAc)₆. The N-acetylglucosaminyltransferase AcetRL-R349H and the application thereof provided by the present invention broaden a product spectrum of N-acetylglucosaminyltransferases, can be used to prepare the CTOS with a high degree of polymerization, and have broad application prospects.

Various terms and phrases used in the present invention have general meanings known to those skilled in the art.

### BRIEF DESCRIPTION OF THE DRAWINGS

FIG. 1: a schematic diagram of HPLC analysis results, where AcetRL represents N-acetylglucosaminyltransferase AcetRL, and AcetRL-R349H represents N-acetylglucosaminyltransferase AcetRL-R349H.

FIG. 2: a comparison diagram of product compositions and yields of fermentation broths, where AcetRL represents N-acetylglucosaminyltransferase AcetRL, and AcetRL-R349H represents N-acetylglucosaminyltransferase AcetRL-R349H.

FIG. 3: a schematic diagram of CavityPlus pocket prediction for N-acetylglucosaminyltransferase AcetRL.

FIG. 4: a 3D diagram of analysis of molecular docking results between N-acetylglucosaminyltransferase AcetRL and chitotetraose.

FIG. 5: a 2D diagram of analysis of molecular docking results between N-acetylglucosaminyltransferase AcetRL and chitotetraose.

FIG. 6: a comparison diagram of product compositions and yields of fermentation broths containing various mutants.

FIG. 7: a 3D diagram of analysis of molecular docking results between N-acetylglucosaminyltransferase AcetRL-R349H and chitopentaose.

FIG. 8: a 2D diagram of analysis of molecular docking results between N-acetylglucosaminyltransferase AcetRL-R349H and chitopentaose.

### DETAILED DESCRIPTION OF THE EMBODIMENTS

The present invention is further described below in conjunction with the examples. However, the scope of the present invention is not limited to the following examples. Those skilled in the art can understand that various changes and modifications can be made to the present invention without departing from the spirit and scope of the present invention.

The instruments, reagents, and materials involved in the following examples, unless otherwise specified, are all conventional instruments, reagents, and materials already available in the prior art and can be obtained through regular commercial means. The experimental methods, detection methods, etc. involved in the following examples, unless otherwise specified, are all conventional experimental methods and detection methods already available in the prior art.

### Example 1 Mining of N-acetylglucosaminyltransferase AcetRL

To mine N-acetylglucosaminyltransferases with product specificity, the present invention screened out a gene (ID: Q8KJI5_RHILI) that was derived from Rhizobium loti and might have an N-acetylglucosaminyltransferase activity by means of Uniprot search and comparison. An amino acid sequence of a protein expressed by the gene was as shown in SEQ ID NO. 1, and it was designated as N-acetylglucosaminyltransferase AcetRL. After Bacillus subtilis-preferred codon optimization, a nucleotide sequence of the gene was as shown in SEQ ID NO. 2.

The amino acid sequence of the N-acetylglucosaminyltransferase AcetRL was as shown in SEQ ID NO. 1, represented as follows:

MTLFATASTVAICSYALLSTVYKTAQVFYTLPTNVPPTSGDPPGGEPW
PSVDVIIPCYNEAPRTLSDCLASIASQEYAGKLQVYVVDDGSANRDAL
VGVHEEYAGDPRFNFIALPKNVGKRKAQIAAIRRSCGDLVLNVDSDTI
LAPDVITRLALKMQDQAVGAAMGQLAASNRSETWLTRLIDMEYWLACN
EERAAQARFGAVMCCCGPCAMYRRSALVSLLDQYETQRFRGKPSDFGE
DRHLTILMLKAGFRTEYVPEAVAATVVPNSMGPYLRQQLRWARSTFRD
TLLAFQLLRGLNIYLTLDVIGQNIGPLLLSLSILAGLAQLVTTGTAPW
TACLMIAAMTIVRCSVAAFRARQLRFLGFSLHTLINIFLLLPLKAYAL
CTLSNSDWLSRSSAANVTDTGGTSSKPNLVGSDAAYSEQQ.

A nucleotide sequence of a coding gene of the N-acetylglucosaminyltransferase AcetRL was as shown in SEQ ID NO. 2, represented as follows (direction 5′-3′):

atgaccctgtttgcaaccgcaagcaccgttgcaatttgtagctatgcactgctgagcaccgtttataaaaccgcaca
ggttttttataccctgccgaccaatgttccgccgaccagcggtgatcctccgggtggtgaaccttggcctagtgttgatgttattat
tccgtgttataacgaagccccgcgtaccctgagcgattgtctggcaagtattgcaagccaggaatatgcaggtaaactgcaggt
ttatgttgtggacgatggtagcgcaaatcgtgatgcactggttggtgttcatgaagaatatgcaggtgatccgcgttttaattttatc
gcactgccgaaaaatgtgggcaaacgtaaagcacagattgccgcaattcgtcgtagctgtggtgatctggttctgaatgttgata
gcgataccattctggcaccggatgttattacccgtctggcactgaaaatgcaggatcaggcagttggtgcagcaatgggtcag
ctggcagcaagcaatcgtagcgaaacctggctgacccgtctgattgatatggaatattggctggcatgtaatgaggaacgtgc
agcacaggcacgttttggtgcagttatgtgttgttgtggtccgtgtgcaatgtatcgtcgtagcgcactggttagcctgctggatc
agtatgaaacccagogttttcgtggtaaaccgagcgattttggtgaagatcgtcatctgaccattctgatgctgaaagcaggtttt
cgtaccgaatatgttccggaagcagttgcagcaaccgttgttccgaatagcatgggtccgtatctgcgtcagcagctgcgttgg
gcacgtagcacatttcgtgataccctgctggcatttcagctgctgcgtggtctgaatatttatctgaccctggatgttattggccag
aatattggtccgctgctgctgagcctgagcattctggcaggtctggcacagctggttaccaccggtaccgcaccttggacagc
atgtttaatgattgcagcaatgaccattgtgcgctgtagcgttgcagcatttcgtgcacgtcagctgcgttttctgggttttagcctg
cataccctgattaatattttcctgctgctgccgctgaaagcatatgcactgtgtaccctgagcaatagcgattggctgagccgtag
cagcgcagcaaatgttaccgataccggtggtaccagcagcaaaccgaatctggttggtagcgatgcagcatatagcgaacag
cagtga.

### Example 2 Construction of a Recombinant Engineering Bacterium Expressing N-acetylglucosaminyltransferase AcetRL

Steps included:
(1) performing whole gene synthesis on genes as shown in SEQ ID NO. 2, and performing PCR amplification to obtain a large number of gene fragments.
(2) Construction of a recombinant expression vector

The gene fragments obtained by the above-mentioned amplification were linked with a pP43NMK cloning vector by using a seamless cloning technology, and linked products were transferred into Escherichia coli (E. coli) DH5a competent cells. Positive transformant screening was performed by using an LB plate containing sodium ampicillin. After clones were verified by colony PCR using primers NMK-F and NMK-R, positive clones were picked for sequencing to obtain a recombinant plasmid.

A nucleotide sequence of the primer NMK-F was as shown in SEQ ID NO. 3, represented as follows (direction 5′-3′):

cgtgtcaaagaaattattgaatcacaaaaaacagg.

A nucleotide sequence of the primer NMK-R was as shown in SEQ ID NO. 4, represented as follows (direction 5′-3′):

gcatgtgggtgatgtagatctcg.

(3) Construction of the recombinant engineering bacterium

A correctly sequenced E. coli DH5a strain was cultured, the recombinant plasmid was extracted and transformed into the Bacillus subtilis strain 168, and a constructed engineering strain was grown on a kanamycin sulfate-resistant plate.
(4) Fermentation of a recombinant engineering strain

Single colonies of the recombinant engineering strain were picked from the plate into an LB medium test tube, cultured for 16 h, then transferred to 50 mL of an M9 medium, and subjected to fermentation culture for 48 h under conditions of 37° C. and 150 rpm; and then fermentation broths were collected.

### Example 3 Determination of Products of N-acetylglucosaminyltransferase AcetRL

The fermentation broths collected in Example 2 were analyzed and were subjected to product identification by using a high-performance liquid chromatography (HPLC). HPLC is a high-performance size exclusion chromatography, equipped with a photodiode array detector (PDA, SPD-M40, Shimadzu). This system adopted a Shodex Asahipak NH2P-50 4E chromatographic column (Shodex) with 70% acetonitrile as a mobile phase and a flow rate of 1.0 mL/min. The fermentation broths were centrifuged (10,000 g, 5 min), and were filtered by a 0.22 μm filter to obtain supernatant; and 10 μL of the supernatant was injected into the HPLC system. Product components were qualitatively determined based on corresponding standard curves.

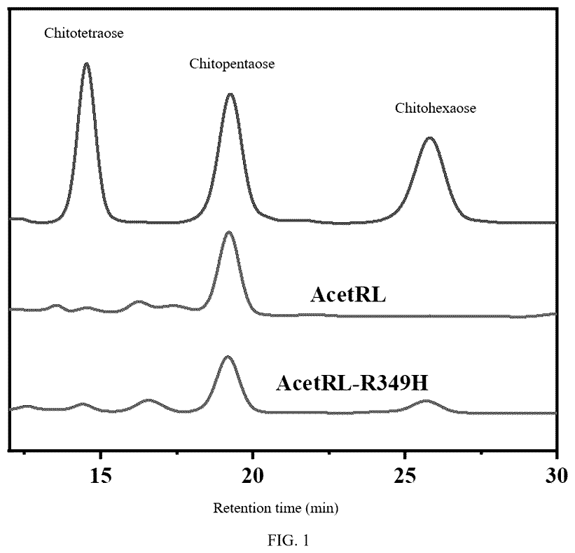

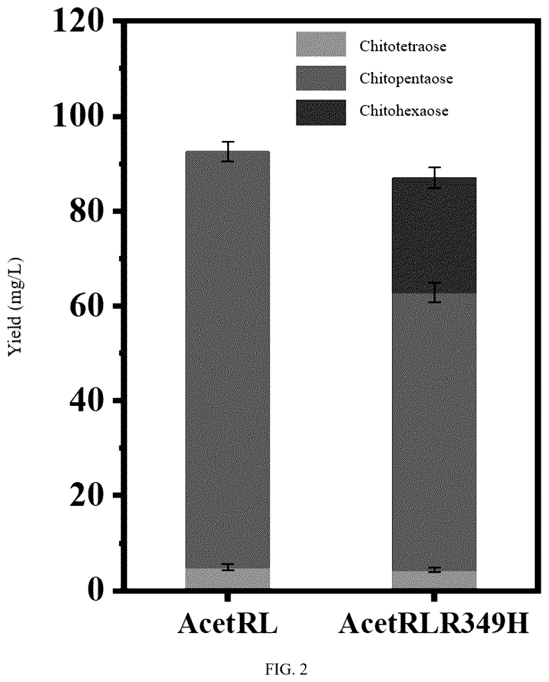

HPLC analysis results were shown in FIG. 1, and comparison of product compositions and yields was shown in FIG. 2. It can be seen that the products of the N-acetylglucosaminyltransferase AcetRL were chitotetraose and chitopentaose.

### Example 4 Modification of N-acetylglucosaminyltransferase AcetRL

As shown by the results of Example 3, the N-acetylglucosaminyltransferase AcetRL can only produce chitotetraose and chitopentaose. Although it has certain product specificity, a product spectrum thereof is insufficiently broad to meet market demands. Therefore, the present invention attempts to perform mutation modification on the N-acetylglucosaminyltransferase AcetRL, in order to obtain N-acetylglucosaminyltransferases that have an ability to produce CTOS with a higher degree of polymerization.

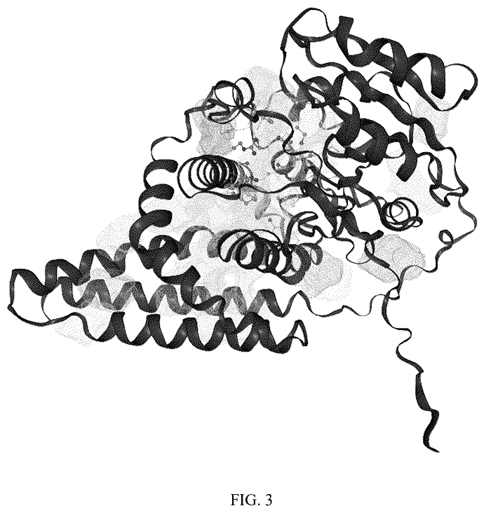

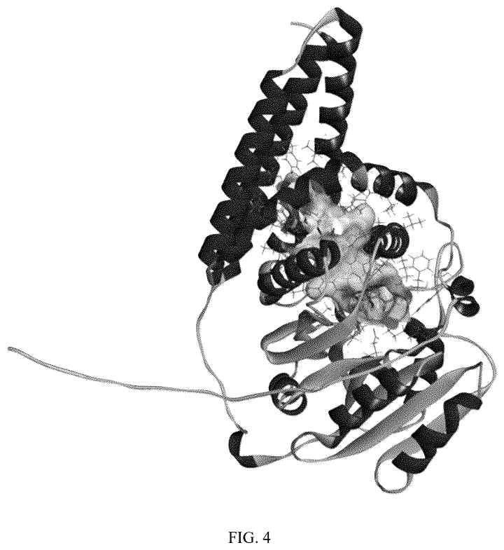

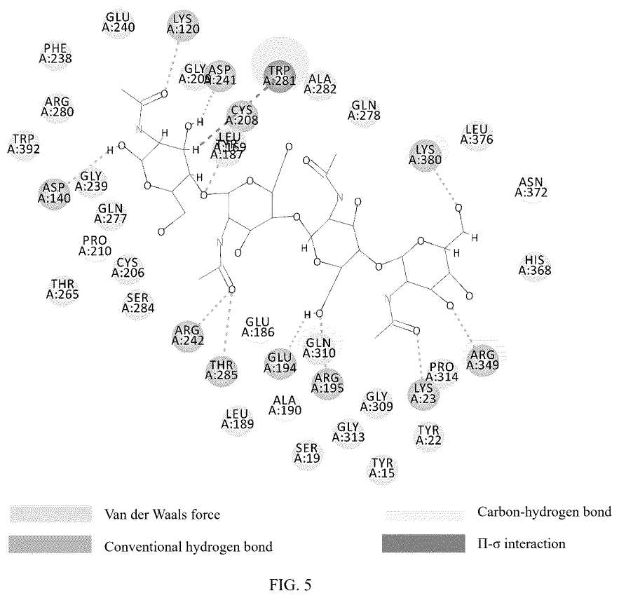

To determine key mutation sites, protein sequences from different sources were comparatively analyzed. AlphaFold 2.0 was used for three-dimensional structure modeling of proteins, and a CavityPlus database was used to perform potential pocket prediction on the N-acetylglucosaminyltransferase AcetRL, with results shown in FIG. 3. Autodock software was used for molecular docking between the substrate (chitotetraose) and an enzyme, and Discovery Studio Client software was used to analyze molecular docking results, with the results shown in FIG. 4 and FIG. 5.

Amino acids at the bottom of a pocket that intersected with amino acids involved in a docking interaction were selected, and based on comparative analysis of N-acetylglucosaminyltransferase protein sequences from different sources, ARG349 was identified as the key site for controlling the degree of polymerization of the products in the N-acetylglucosaminyltransferase AcetRL. Therefore, the present invention selected to perform saturation mutagenesis on the ARG349 site, yielding 19 mutants temporarily designated as: AcetRL-R349G, AcetRL-R349A, AcetRL-R349V, AcetRL-R349L, AcetRL-R349I, AcetRL-R349M, AcetRL-R349F, AcetRL-R349W, AcetRL-R349P, AcetRL-R349S, AcetRL-R349T, AcetRL-R349C, AcetRL-R349Y, AcetRL-R349N, AcetRL-R349Q, AcetRL-R349D, AcetRL-R349E, AcetRL-R349K, AcetRL-R349H.

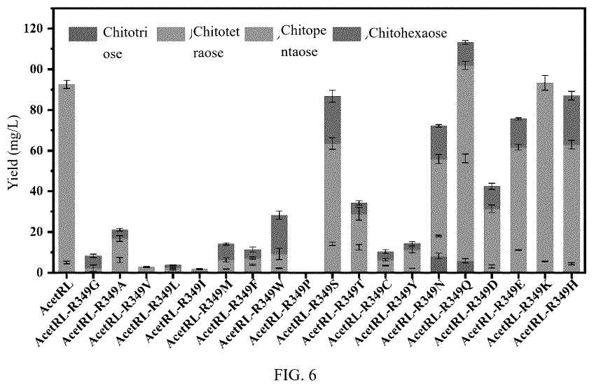

19 mutant plasmids were constructed by a conventional method and respectively transformed into the Bacillus subtilis strain 168 for heterologous expression (with the method as described in Example 2), yielding fermentation broths containing various mutants. The products of each fermentation broth were determined according to the method described in Example 3 (using the fermentation broths from Example 2 as control), with results shown in FIG. 6. As shown in FIG. 6, a plurality of mutants had the ability to produce chitohexaose, but the levels of their ability varied significantly. Among them, AcetRL-R349Q showed the highest total CTOS yield, which was 113.78 mg/L, but its chitohexaose yield was only 11.35 mg/L. AcetRL-R349S and AcetRL-R349H showed the highest chitohexaose yields, reaching 23.32 mg/L and 24.07 mg/L, respectively. However, AcetRL-R349H showed a chitopentaose yield of 58.55 mg/L, which was greater than a chitopentaose yield (49.47 mg/L) of AcetRL-R349S. Therefore, AcetRL-R349H exhibited a superior ability to produce CTOS with a high degree of polymerization compared to AcetRL-R349S. The present invention selected AcetRL-R349H for subsequent studies and designated this mutant as N-acetylglucosaminyltransferase AcetRL-R349H.

A construction process of the N-acetylglucosaminyltransferase AcetRL-R349H was as follows:

An amino acid at a 349th site of the N-acetylglucosaminyltransferase AcetRL was changed from arginine to histidine, with a specific method including: performing site-directed mutagenesis on the N-acetylglucosaminyltransferase AcetRL at a residue ARG349, and changing a codon corresponding to the amino acid at the 349th site on a coding gene of the N-acetylglucosaminyltransferase AcetRL from a codon “CGC” of arginine to a codon “CAT” of histidine. An amino acid sequence of the N-acetylglucosaminyltransferase AcetRL-R349H was as shown in SEQ ID NO. 5, and a nucleotide sequence of the coding gene thereof was as shown in SEQ ID NO. 6.

The amino acid sequence of the N-acetylglucosaminyltransferase AcetRL-R349H was as shown in SEQ ID NO. 5, represented as follows:

MTLFATASTVAICSYALLSTVYKTAQVFYTLPTNVPPTSGDPPGGEPWP
SVDVIIPCYNEAPRTLSDCLASIASQEYAGKLQVYVVDDGSANRDALVG
VHEEYAGDPRFNFIALPKNVGKRKAQIAAIRRSCGDLVLNVDSDTILAP
DVITRLALKMQDQAVGAAMGQLAASNRSETWLTRLIDMEYWLACNEERA
AQARFGAVMCCCGPCAMYRRSALVSLLDQYETQRFRGKPSDFGEDRHLT
ILMLKAGFRTEYVPEAVAATVVPNSMGPYLRQQLRWARSTFRDTLLAFQ
LLRGLNIYLTLDVIGQNIGPLLLSLSILAGLAQLVTTGTAPWTACLMIA
AMTIVHCSVAAFRARQLRFLGFSLHTLINIFLLLPLKAYALCTLSNSDW
LSRSSAANVTDTGGTSSKPNLVGSDAAYSEQQ.

The nucleotide sequence of the coding gene of the N-acetylglucosaminyltransferase AcetRL-R349H was as shown in SEQ ID NO. 6, represented as follows (direction 5′-3′):

atgaccctgtttgcaaccgcaagcaccgttgcaatttgtagctatgcactgctgagcaccgtttataaaaccgcaca
ggttttttataccctgccgaccaatgttccgccgaccagcggtgatcctccgggtggtgaaccttggcctagtgttgatgttattat
tccgtgttataacgaagccccgcgtaccctgagcgattgtctggcaagtattgcaagccaggaatatgcaggtaaactgcaggt
ttatgttgtggacgatggtagcgcaaatcgtgatgcactggttggtgttcatgaagaatatgcaggtgatccgcgttttaattttatc
gcactgccgaaaaatgtgggcaaacgtaaagcacagattgccgcaattcgtcgtagctgtggtgatctggttctgaatgttgata
gcgataccattctggcaccggatgttattacccgtctggcactgaaaatgcaggatcaggcagttggtgcagcaatgggtcag
ctggcagcaagcaatcgtagcgaaacctggctgacccgtctgattgatatggaatattggctggcatgtaatgaggaacgtgc
agcacaggcacgttttggtgcagttatgtgttgttgtggtccgtgtgcaatgtatcgtcgtagcgcactggttagcctgctggatc
agtatgaaacccagcgttttcgtggtaaaccgagcgattttggtgaagatcgtcatctgaccattctgatgctgaaagcaggtttt
cgtaccgaatatgttccggaagcagttgcagcaaccgttgttccgaatagcatgggtccgtatctgcgtcagcagctgcgttgg
gcacgtagcacatttcgtgataccctgctggcatttcagctgctgcgtggtctgaatatttatctgaccctggatgttattggccag
aatattggtccgctgctgctgagcctgagcattctggcaggtctggcacagctggttaccaccggtaccgcaccttggacagc
atgtttaatgattgcagcaatgaccattgtgcattgtagcgttgcagcatttcgtgcacgtcagctgcgttttctgggttttagcctg
cataccctgattaatattttcctgctgctgccgctgaaagcatatgcactgtgtaccctgagcaatagcgattggctgagccgtag
cagcgcagcaaatgttaccgataccggtggtaccagcagcaaaccgaatctggttggtagcgatgcagcatatagcgaacag
cagtga.

The entire plasmid (i.e., a pP43NMK plasmid of the coding gene of the N-acetylglucosaminyltransferase AcetRL) was then subjected to PCR amplification. PCR products were treated with a restriction enzyme DpnI to digest a methylated parental template, thereby obtaining a modified plasmid. Nucleotide sequences of specific primers used for the PCR amplification were as shown in SEQ ID NO. 7 and SEQ ID NO. 8.

A nucleotide sequence of a forward primer was as shown in SEQ ID NO. 7, represented as follows (direction 5′-3′):

cattgtgcattgtagcgttgcagcatttcg.

A nucleotide sequence of a reverse primer was as shown in SEQ ID NO. 8, represented as follows (direction 5′-3′):

caacgctacaatgcacaatggtcattgctgc.

### Example 5 Determination of Products of N-acetylglucosaminyltransferase AcetRL-R349H

A recombinant engineering bacterium expressing the N-acetylglucosaminyltransferase AcetRL-R349H was constructed and fermented. Collected fermentation broths were analyzed with the method as described in Example 3. HPLC analysis results were shown in FIG. 1, and comparison of product compositions and yields was shown in FIG. 2. It can be seen that the products of the N-acetylglucosaminyltransferase AcetRL-R349H included chitohexaose, with a yield as high as 24.07 mg/L, which was not achievable by the N-acetylglucosaminyltransferase AcetRL.

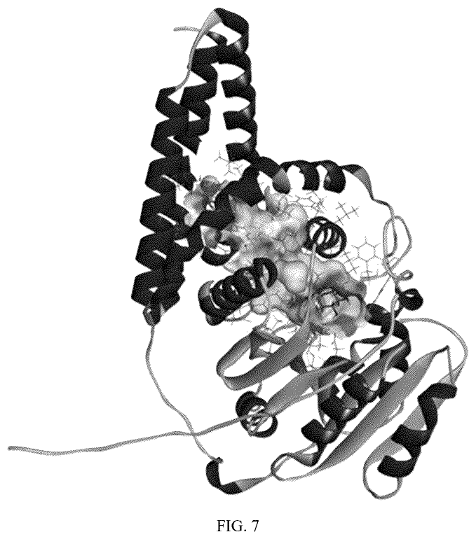

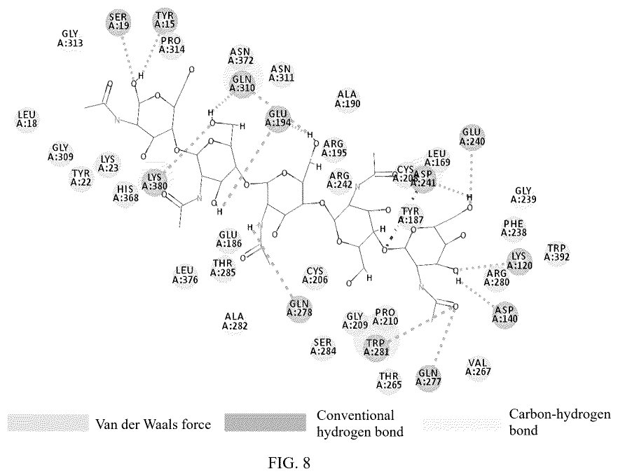

AlphaFold 2.0 was used to further construct a structural model of the N-acetylglucosaminyltransferase AcetRL-R349H, and molecular docking with chitopentaose was performed using Autodock, with results shown in FIG. 7 and FIG. 8. A maximum product of N-acetylglucosaminyltransferase AcetRL docking was chitotetraose, whereas the N-acetylglucosaminyltransferase AcetRL-R349H could achieve docking with chitopentaose. The N-acetylglucosaminyltransferase AcetRL-R349H allowed the insertion of chitopentaose, which might be the basis for the production of chitohexaose; and meanwhile, an extremely strong hydrogen bond interaction between amino acid residues at the entrance of an active pocket and a ligand might be another key factor for the production of chitohexaose.

The foregoing examples are provided for those skilled in the art to fully disclose and describe how the claimed embodiments are implemented and used, rather than to limit the scope of the disclosure herein. Modifications that would be obvious to those skilled in the art shall fall within the scope of the appended claims.

What is claimed is:

1. An N-acetylglucosaminyltransferase AcetRL-R349H, wherein an amino acid sequence of the N-acetylglucosaminyltransferase AcetRL-R349H is as shown in SEQ ID NO. 5.

2. An application of the N-acetylglucosaminyltransferase AcetRL-R349H according to claim 1 in preparation of CTOS, wherein the CTOS are any one or two or more of chitotetraose, chitopentaose, or chitohexaose.

3. The application of the N-acetylglucosaminyltransferase AcetRL-R349H in preparation of CTOS according to claim 2, wherein in specific applications, a coding gene of the N-acetylglucosaminyltransferase AcetRL-R349H is introduced into Bacillus subtilis to construct a recombinant engineering bacterium expressing the N-acetylglucosaminyltransferase AcetRL-R349H; and the recombinant engineering bacterium is fermented to obtain a fermentation broth containing the CTOS.

4. The application of the N-acetylglucosaminyltransferase AcetRL-R349H in preparation of CTOS according to claim 3, wherein the Bacillus subtilis is a Bacillus subtilis strain 168.
